(19) United States
(12) Patent Application Publication
(10) Pub. No.: US 2012/0239078 A1
(43) Pub. Date: Sep. 20, 2012

(54) QUICK RELEASE TOURNIQUET

(76) Inventors: Sandy Maney, Waynesville, NC (US); Velda Biddix, Marion, NC (US); Karen Johnson, Anderson, SC (US)

(21) Appl. No.: 13/048,998

(22) Filed: Mar. 16, 2011

(51) Int. Cl. A61B 17/132

(52) U.S. Cl. 606/203

(57) ABSTRACT

A tourniquet with a generally hexagonally shaped upper frame. The upper frame has a linear upper frame elements meeting at an upper apex. Linear side frame elements meet a linear upper frame element at an upper side apex. Linear lower frame elements meets one side linear side frame element at a lower side apex wherein the linear lower frame elements extend beyond an overlapping apex. Each linear lower frame element of said linear lower frame elements comprises a terminus with a handle attached to each terminus.

### BACKGROUND

The present invention is directed to a tourniquet which is suitable for use in multiple and diverse applications. More specifically, the present invention is related to a tourniquet which allows for use in multiple applications and is quickly applied and released.

Tourniquets have been widely used in the medical arts for such applications as compressing an underlying blood vessel to securing a drape in the vicinity of a medical procedure site. There are a myriad of tourniquets from as simple as a flexible tube which is secured around the arm to complex bladder type tourniquets as commonly employed in a blood pressure cuff. Many tourniquets have closure mechanisms such as snaps, VELCRO®, various engaging members and the like which are cumbersome to use, especially, while wearing gloves as is most often the case.

Of particular relevance to the present invention are tourniquets made from materials with spring type properties which can be manipulated to be placed on an appendage and then allowed to relax to a near rest shape. These have previously been considered inferior due to the inability to control the relaxation and they can not be placed in a way to selectively collapse blood vessels or not collapse blood vessels.

There is an ongoing need for an improve tourniquet which is devoid of the deficiencies of the art. The present invention provides such a device which is easily manipulated for quick release.

### SUMMARY

It is an object of the invention to provide a quick release tourniquet.

It is another object of the invention to provide an improved tourniquet which can be used in multiple and diverse applications.

An advantage of the inventive tourniquet is the ease of manufacturing and use.

These and other advantages, as will be realized, are provided in a tourniquet. The tourniquet has a generally hexagonally shaped upper frame. The upper frame has a linear upper frame elements meeting at an upper apex. Linear side frame elements meet a linear upper frame element at an upper side apex. Linear lower frame elements meets one side linear side frame element at a lower side apex wherein the linear lower frame elements extend beyond an overlapping apex. Each linear lower frame element of said linear lower frame elements comprises a terminus with a handle attached to each terminus.

Yet another embodiment is provided in a method of applying a tourniquet to an appendage. The method includes providing a tourniquet wherein the tourniquet has a generally hexagonally shaped upper frame with linear upper frame elements meeting at an upper apex; linear side frame elements wherein each linear side frame element of the linear side frame elements meets a linear upper frame element of the linear upper frame elements at an upper side apex; linear lower frame elements wherein each linear lower frame element of the linear lower frame elements meets one said side linear side frame element at a lower side apex wherein the linear lower frame elements extend beyond an overlapping apex wherein each linear lower frame element of the linear lower frame elements each comprises a terminus; and handles wherein a handle of said handles is attached to each terminus. The method further includes grasping each handle and persuading the handles away from each other thereby forming a passage between the termini sufficiently large to pass the appendage through the passage. The appendage is passed through the passage. The handles are allowed to decrease the passage to secure the appendage within the generally hexagonally shaped upper frame.

### BRIEF DESCRIPTION OF FIGURES

FIG. 1 is a schematic representation of an embodiment of the invention.

FIG. 2 is a front schematic view of an embodiment of the invention.

FIG. 3 is a side schematic view of the embodiment shown in FIG. 2.

FIGS. 4A-4D illustrate an embodiment of the invention in schematic form.

### DETAILED DESCRIPTION

The present invention is related to an improved tourniquet. More specifically, the present invention is related to a tourniquet that can be easily reversibly placed on an appendage of a patient without requiring manipulation of mating components.

The invention will be described with reference to the various figures which are an integral, non-limiting, component of the instant disclosure. Throughout the various figures similar elements will be numbered accordingly.

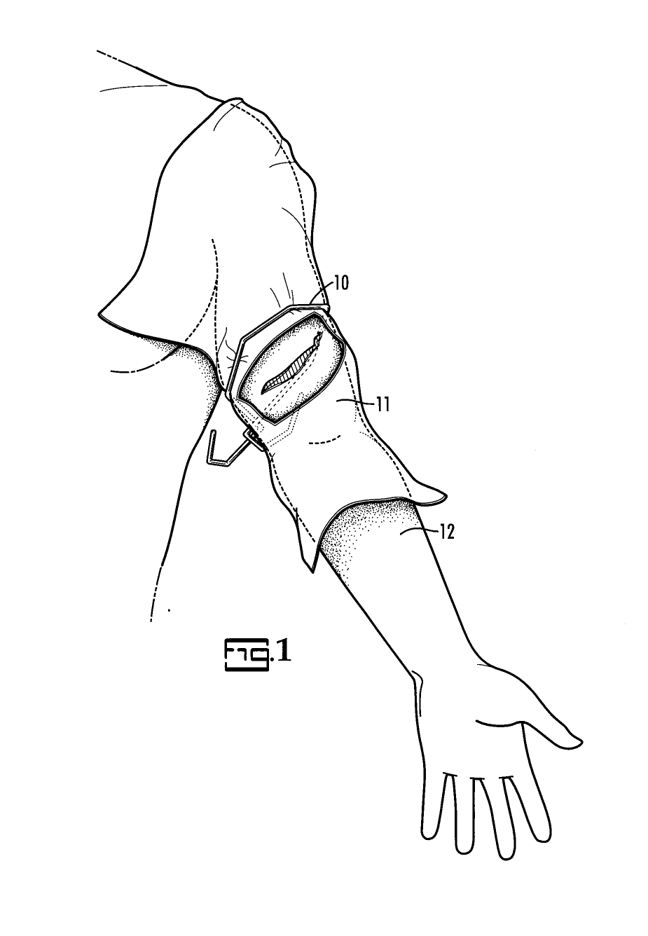

An embodiment of the invention will be described with reference to FIG. 1 wherein a tourniquet, 10, is shown around an appendage, 12, represented as a human arm. While illustrated as an arm the appendage could be an arm, leg, thumb, finger or toe. In one embodiment the tourniquet can be used to secure a surgical drape, 11, in proper position during a surgical procedure.

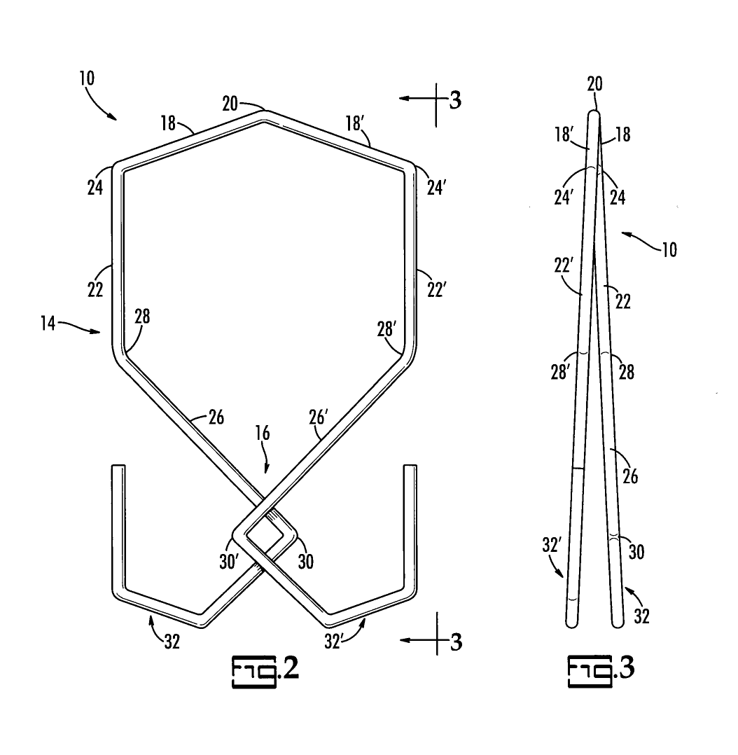

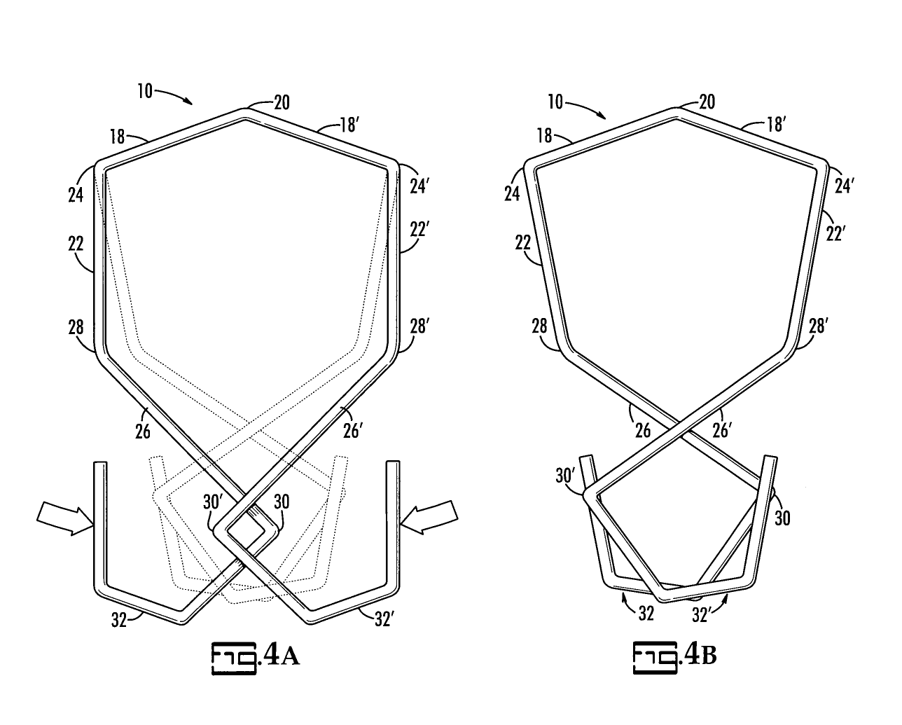

A tourniquet in accordance with an embodiment of the invention is illustrated in front view in FIG. 2 and side view, taken along line 3-3 of FIG. 2, in FIG. 3. The tourniquet, 10, comprises a generally hexagonally shaped upper frame, 14, with an overlapping apex, 16, which is not connected. The generally hexagonally shaped upper frame comprises linear upper frame elements, 18 and 18′, meeting at an upper apex, 20. Linear side frame elements, 22 and 22′, each meets the linear upper frame elements, 18 and 18′, at an upper side apex, 24 and 24′. Linear lower frame elements, 26 and 26′, each meets the linear side frame elements, 22 and 22′, at a lower side apex, 28 and 28′. The linear lower frame elements 26 and 26′, extend beyond the overlapping apex, 16, to a terminus, 30 and 30′.

Handles, 32 and 32′, are attached to the terminus of the linear lower frame elements, 26 and 26′. The purpose of the handles will be further understood from disclosure herein. The handles preferably comprise linear portions generally in the shape of a vacated pentagon or vacated circle wherein the term “vacated” indicates one linear portion, in the case of a pentagon, or one arc, in the case of a circle is absent. A generally pentagon shape is preferred due to simplicity in manufacturing. In one embodiment the handle may be continuous and integral to the furthest extent of the handle to the linear lower frame elements 26 and 26′. A continuous, or closed, handle is not preferred due to the additional manufacturing steps required to accomplish the attachment.

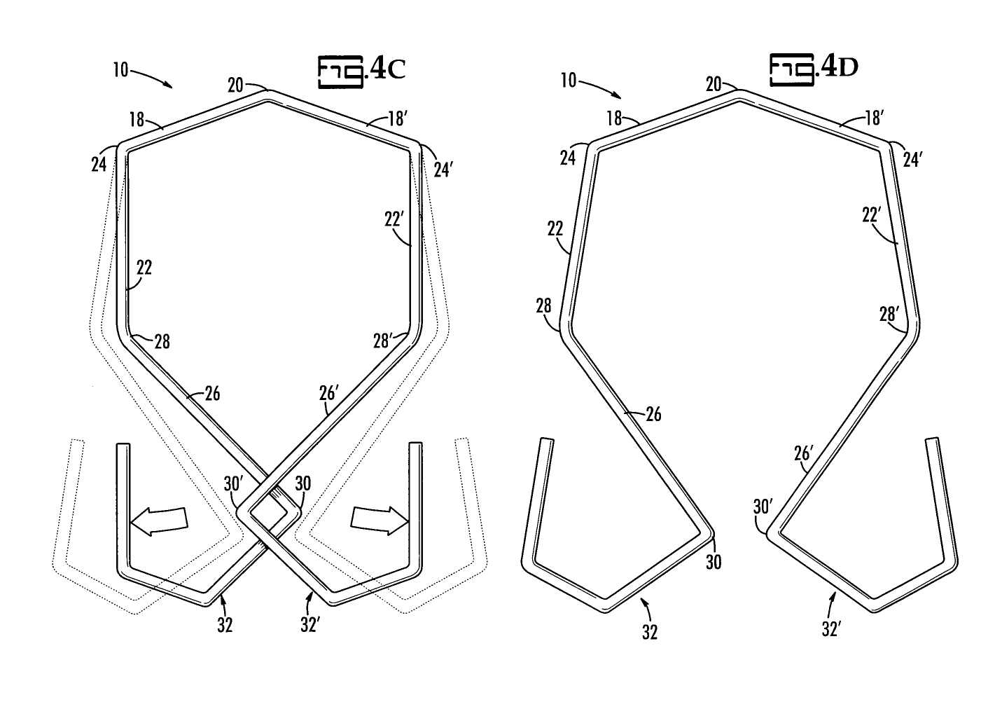

An embodiment of the invention will be described with reference to FIGS. 4A-4D. The tourniquet is illustrated in a rest shape in FIG. 4B. In use, the technician will grasp the handles, 32 and 32′, and draw the handles apart as represented by the arrows in FIG. 4C, thereby forming a passage, 34, between the terminus, 30 and 30′, of the linear lower frame elements, 26 and 26′. The handles are drawn apart to the extent necessary for the appendage to pass through the passage, 34, at which point the tourniquet is allowed to return towards rest shape as indicated by the arrows of FIG. 4A. As would be readily realized the extent to which the tourniquet relaxes towards rest shape is dependent on the size of the appendage contained therein.

An advantage of the tourniquet can be realized by examination of FIGS. 4A-4D. Round tourniquets have a disadvantage that when they are opened the entire ring is expanded evenly. Therefore, upon return to rest shape the ring changes size symmetrically. With the present invention, the primary bending occurs at the lower side apex, 28 and 28′, and upper side apex, 24 and 24′, with minimal distortion at the upper apex, 20. This is advantageous, particularly when used with a drape, which is a cloth laid over a portion of the patient, since the drape can be held down by the linear upper frame elements, 18 and 18′, and the tourniquet can be allowed to relax without the drape becoming bunched under the tourniquet. Yet another advantage of the instant invention is the ability to place an apex in a position wherein blood flow is not restricted or a linear frame element can be specifically placed in a location wherein blood flow is restricted. This provides additional utilities not currently available.

The linear upper frame elements, linear side frame elements and linear lower frame elements are independently at least 0.5 inches to 6 inches and more preferably at least 1 inch to 3 inches in length.

It is most preferred that the tourniquet is a continuous element bent into the shape as illustrated. Most preferably, the continuous element has a rounded cross-sectional shape although other cross-sectional shapes include square, pentagonal, hexagonal, etc. may be employed. A round cross-section is preferred due to the extensive availability of round materials which are suitable for use in the invention. Symmetrical shapes are preferred due to fact that symmetrical cross-sections are typically more readily extruded. In one embodiment the tourniquet comprises a central material with a non-allergenic coating thereon.

The material of construction is not particularly limiting with the proviso that the material has sufficient strength to maintain the basic shape when repeatedly distorted to place on the appendage and returns to rest shape, as represented in FIG. 2 or 4B, when released. Rest shape is the shape which the tourniquet is in when distorting forces are not applied.

Geometrical shapes such as hexagon, pentagon, circular, etc. or their equivalent terms such as hexagonal, pentagonal, round, circular, etc. are intended to include regular geometries and irregular geometries. Adjacent linear members may be the same length or a different length with the difference between adjacent linear members having a length which is at least 50% to no more than 150% relative to each other.

The invention has been described with reference to preferred embodiments without limit thereto. One of skill in the art would realize additional limitations and advantages which are not specifically enumerated but which are within the scope of the invention as more specifically set forth in the claims appended hereto.

Claimed is:

1. A tourniquet comprising:
a generally hexagonally shaped upper frame comprising:
linear upper frame elements meeting at an upper apex;
linear side frame elements wherein each linear side frame element of said linear side frame elements meets a linear upper frame element of said linear upper frame elements at an upper side apex;
linear lower frame elements wherein each linear lower frame element of said linear lower frame elements meets one said side linear side frame element at a lower side apex wherein said linear lower frame elements extend beyond an overlapping apex wherein each linear lower frame element of said linear lower frame elements comprises a terminus; and
handles wherein a handle of said handles attached to each terminus.

2. The tourniquet of claim 1 wherein said handle comprises linear portions.

3. The tourniquet of claim 1 wherein said handle is in a shape selected from vacated pentagon and vacated circle.

4. The tourniquet of claim 1 wherein said linear upper frame element, said linear side frame element, said linear lower frame element each independently has a length of at least 0.5 inches to no more than 6 inches.

5. The tourniquet of claim 4 wherein said linear upper frame element, said linear side frame element, said linear lower frame element each independently has a length of at least 1 inches to no more than 3 inches.

6. A method of applying a tourniquet to an appendage comprising:
providing a tourniquet comprising:
a generally hexagonally shaped upper frame comprising:
linear upper frame elements meeting at an upper apex;
linear side frame elements wherein each linear side frame element of said linear side frame elements meets a linear upper frame element of said linear upper frame elements at an upper side apex;
linear lower frame elements wherein each linear lower frame element of said linear lower frame elements meets one said side linear side frame element at a lower side apex wherein said linear lower frame elements extend beyond an overlapping apex wherein each linear lower frame element of said linear lower frame elements each comprises a terminus; and
handles wherein a handle of said handles is attached to each terminus;
grasping each handle and persuading said handles away from each other thereby forming a passage between said termini sufficiently large to pass said appendage through said passage;
passing said appendage through said passage; and
allowing said handles to decrease said passage to secure said appendage within said generally hexagonally shaped upper frame.

7. The method of applying a tourniquet to an appendage of claim 6 wherein said handle comprises linear portions.

8. The method of applying a tourniquet to an appendage of claim 6 wherein said handle is in a shape selected from vacated pentagon and vacated circle.

9. The method of applying a tourniquet to an appendage of claim 6 wherein said linear upper frame element, said linear side frame element, said linear lower frame element each independently has a length of at least 0.5 inches to no more than 6 inches.

10. The method of applying a tourniquet to an appendage of claim 9 wherein said linear upper frame element, said linear side frame element, said linear lower frame element each independently has a length of at least 1 inches to no more than 3 inches.

11. The method of applying a tourniquet to an appendage of claim 6 further comprising:
providing a surgical drape.

12. The method of applying a tourniquet to an appendage of claim 11 further comprising:
placing said surgical drape on said appendage and placing said securing said surgical drape to said appendage by said tourniquet.
